(19) United States
(12) Patent Application Publication
(10) Pub. No.: US 2003/0233126 A1
(43) Pub. Date: Dec. 18, 2003

(54) Injection devices and methods for testing implants

(75) Inventors: Hilton M. Kaplan, Beverly Hills, CA (US); Gerald E. Loeb, South Pasadena, CA (US)

Correspondence Address: MCDERMOTT, WILL & EMERY, Suite 3400, 2049 Century Park East, Los Angeles, CA, 90067, US

(73) Assignees: Alfred E. Mann Institute for Biomedical Engineering; at the University of Southern California

(21) Appl. No.: 10/461,560

(22) Filed: Jun. 12, 2003

Related U.S. Application Data
(60) Provisional application No. 60/388,370, filed on Jun. 12, 2002.
(60) Provisional application No. 60/476,007, filed on Jun. 4, 2003.

(51) Int. Cl. A61N 1/18

(52) U.S. Cl. 607/3; 604/57

(57) ABSTRACT

The invention relates to devices and methods for positioning an implant at a target location in the body. The methods include testing an implant while within an injection device at a target location to determine whether the implant is functioning effectively. The methods also include testing from or delivery of materials to the target location during implantation, and loading the injection device for use. The device may be configured to permit the longitudinal and/or axial position of the implant to be maintained relative to an injection device during implantation. The device may also be configured to permit testing of the implant. The invention also may include implants configured for use in the injection devices.

### CROSS-REFERENCE TO RELATED APPLICATIONS

This application claims priority from U.S. Provisional Patent Application No. 60/388,370, filed, Jun. 12, 2002, and U.S. Provisional Patent Application 60/XXX,XXX, entitled, “Cargo Delivery Capsule: Method and Apparatus for Precise and Protected Delivery of Cargo into Body Tissues and Cavities,” filed Jun. 4, 2003 (Inventor—Hilton M. Kaplan; Attorney Docket No. 64693-066), which are herein incorporated by reference in their entirety.

### BACKGROUND OF THE INVENTION

1. Field of the Invention

This application is related to devices and methods for positioning an implant in a body at a target location at which the implant will function effectively. The application is also related to implants modified for positioning using such devices. Finally, the application is related to methods for loading devices.

2. General Background and State of the Art

It has become desirable to position implants with a high degree of accuracy into specific locations in the body to achieve various physiologic goals. However, positioning implants into target locations of the body may be a difficult task. It may be desirable to position implants using the least invasive method possible to minimize discomfort and risk of infection to the patient generally. It may also desirable to keep implant size relatively small so as not to interfere with the patient's daily activity and to minimize tissue trauma at the target location in which the implant is to be positioned. However, it may also be desirable to maximize the accuracy of implant positioning relative to the target location so that the implant achieves the desired physiological result. For example, microstimulators may be implanted in the proximity of a nerve or muscle to supplement or replace function. More specifically, the rotational orientation of the implant with respect to the body part may also be important to its function. For example, an accelerometer may be implanted that senses the directional force of gravity and motion on the body part.

Various devices and methods are known for positioning implants in the body. In one method, implant positioning may be undertaken by interventive radiologists who position the implant by visualizing the implant relative to the target location using fluoroscopic, CT-guided or ultrasonic imaging for example. In this method, the delivery device or implant contained therein must be constructed of or include an x-ray opaque marker such that the position of the implant can be detected in the x-ray image. While this technique facilitates accurate anatomical placement of an implant, this technique may have several disadvantages. First, this technique allows only for the for the testing of the target site by a temporary stimulator which may not be placed in the same position as the implant. Second, this technique may require that the radiologist and patient be exposed to radiation to visualize the implant.

In a second method, implant positioning may be achieved by first inserting a trochar surrounded by an outer plastic sheath into the body. A conductive distal tip of the trochar may be used to electrically stimulate a test location to evoke a response. The trochar/outer sheath assembly may be moved and electrical stimulation may be repeated until the desired response is achieved. The trochar may then be removed from the outer plastic sheath while holding the sheath in position in the body. An implant may then be manually inserted into the outer sheath and pushed out past the outer sheath distal end with an inner blunt push rod. The outer sheath and push rod may then be removed from the patient leaving the implant behind.

While this technique allows for functional testing of the target location with the outer sheath distal tip, this technique may have several disadvantages. First, this technique allows only for the for the testing of the target site by a temporary stimulator which may not be placed in the same position as the implant. Second, this technique does not permit highly accurate longitudinal placement of the implant relative to the test location, as the position of the outer sheath tip differs from that of the conductive distal tip of the trochar which must protrude from the outer sheath tip to be used for the electrical stimulation testing, and also because the implant itself may be pushed out beyond the outer sheath distal tip to reach its final position. Third, this technique may not permit highly accurate axial orientation of a directionally functional implant. Fourth, this technique may require patient repositioning where retrograde/upward implant positioning may be required relative to the patient, as the implant has a tendency to slide out of the outer sheath when held in a downward position. Fifth, this technique may require handling of the implant during the implantation process which may effect sterility of the method. Sixth, handling of the implant and pushing the implant through the outer sheath and into the tissue may cause damage to the implant itself. Seventh, the use of a beveled needle to deliver the implant to the target location may cause tissue damage at the target location as the needle bevel can slice tissues, such as small nerves and vessels, as the needle distal tip is positioned or repositioned within the target location. Finally, during the manipulations required to remove the trochar and insert and eject the implant, there may be a high risk that the insertion tool will drift in the body so that the implant winds up in a different location than intended.

In a variant of the second method, one end of an elongated cylindrical implant may be wedged into the end of a plastic inner sheath. When the trochar is removed from the outer sheath, the assembly consisting of the implant and inner sheath may be inserted in its place, leaving the implant protruding from the end of the outer sheath but still captured in the end of the inner sheath. In this position, it may be possible to activate the implant for testing purposes and to make small adjustments in position, such as decreasing depth. If the location is judged acceptable, the implant may be extruded from the end of the inner sheath by a blunt push rod located within the inner sheath and the entire insertion tool (outer sheath, inner sheath and push rod) may be removed from the body. If the location is not acceptable, the assembly consisting of the implant and inner sheath may be removed from the outer sheath and replaced by the sharp trochar before any significant repositioning of the insertion tool can be attempted. This method may share most of the disadvantages articulated for the method described above, in particular the tendency for the insertion tool to drift during the manipulations which may be used to replace the trochar with the implant and the ejection of the implant into the body. The outside diameter of the insertion tool may also tends to be somewhat larger because it may accommodate the sum of the implant diameter, the wall thickness of the inner sheath plus the wall thickness of the outer sheath.

### INVENTION SUMMARY

Accordingly, a need remains for an injection device, implants and methods of use to address all of the above stated disadvantages of the known devices and methods.

One objective of the present invention is the development of an injection device for the highly accurate positioning of small implants in the body. Another objective is highly accurate orientation of an implant in longitudinal and/or axial orientation relative to a target location. Another objective is functional testing of the implant at a target location prior to release from the injection device. Another objective is the ability to retrieve the implant prior to implant release if so desired.

Another objective is delivery of an implant to a target site without handling by the user to maximize the sterility of the procedure and minimize damage to the implant. Another objective is to provide structural protection of the implant during delivery to a target location to minimize the loss of or damage to the implant during injection. Another objective is to provide structural protection to minimize the insertion force on the implant. Another objective is to minimize tissue trauma at the target location during implantation.

Another objective is pre-testing or treatment of the target location prior to implant release or post-testing or treatment of the target location after implant release to enhance the likelihood that the implant will have the desired effect in the target tissue.

Another objective is to provide an injection device, implants and methods which can be utilized in combination with other known devices or methods used in implant positioning.

In one embodiment, the invention may include a method for positioning an implant in a body at a target location at which the implant will function effectively including: (a) inserting a distal tip of a cannula having the implant retained in the cannula lumen into the body until the implant reaches a testing position; (b) testing the implant while within the cannula lumen at the testing position to determine whether the implant is functioning effectively; (c) discharging the implant from the lumen of the cannula at the testing location if the testing reveals that the implant is functioning effectively at the test location. This method may be utilized to pre-test the implant itself at the target location prior to releasing it from the injection device.

In one embodiment, the invention may include a method for positioning an implant in a body at a target location at which the implant will function effectively including: (a) inserting a distal tip of a cannula having the implant retained within a cannula lumen into the body until the tip reaches a testing position; (b) withdrawing material from the testing position through a lumen extending from a distal end of a cannula proximate to the testing position to a proximal end of the cannula; (c) testing the material withdrawn from the testing position; (d) discharging the implant from the cannula lumen at the testing position if the testing shows that the implant will operate effectively at the test location. This method may be utilized to test the environment at the target location prior to releasing the implant from the injection device.

In one embodiment, the invention may include a method for injecting material at the site of an implant in a body, including: (a) inserting a distal tip of a cannula having an implant retained within a cannula lumen to a site within the body; (b) delivering material to the area of the site through a lumen extending from a proximal end to a distal end of a cannula; and (c) discharging the implant from the cannula lumen at the site. The implant may be discharged, and material delivered to the site after the material is discharged. This method may be used to treat the target location prior to or after implant positioning.

In one embodiment, the invention may include a method of loading an implant having an implant end, into an injection device including a cannula, and a probe having a distal end sized to fit within the cannula, the method including: (a) inserting the distal end of the probe within the cannula lumen; (b) abutting the implant end against the probe distal end; and (c) moving the cannula relative to the probe until the cannula substantially covers the implant without allowing the implant end to separate from the probe distal end.

In one embodiment, the injection device may include a cannula, an implant having at least one implant external electrode positioned within the cannula lumen; and a channel in the cannula wall substantially aligned with the implant external electrode. This embodiment may be utilized to pre-test the effectiveness of an implant at a target location prior to releasing the implant from the cannula by permitting interstitial fluid at the target location to contact the implant electrode.

In one embodiment, the injection device may include a cannula having a lumen, and an implant positioned within the cannula lumen, such that an end surface of an implant is configured to releasably engage a surface within the cannula lumen. This embodiment may be utilized to prevent longitudinal movement of the implant relative to the injection device during implantation.

In one embodiment, the injection device may include a cannula, a probe and implant positioned in the cannula lumen, such that an implant end surface abuts the probe distal end surface. Both the implant and probe distal end surfaces may be configured to prevent the implant from rotating with respect to the probe while the surfaces abut. This embodiment may be utilized to prevent axial rotation of the implant relative to the injection device during implantation.

In one embodiment, the invention may include an implant configured to be injected by an injection device into body tissue or a body cavity and configured with a surface that interlocks with a surface in the injection device. This embodiment may be used to restrict axial rotation and/or longitudinal movement of the implant relative to the injection device during implantation. This embodiment may further include implants, such as a capsule containing bioactive materials, wherein the capsule dissolves after being injected in the target location to free the material therein.

In one embodiment, the injection device may include a housing containing a material that will not shield/interfere with electromagnetic signals and/or an electrically insulating material that is configured to house the implant while the injection device is being inserted into the body. This embodiment may be utilized for pre-testing implants which communicate using electromagnetic radiation and/or electric current at a target position before release from the injection device.

In one embodiment, the injection device may include a cannula having a cannula distal end formed into a trochar and an implant releasably engaged within the cannula. This embodiment may further include an apparatus for releasing the implant from the cannula lumen into the body at a target location. This embodiment may be utilized to protect the implant within the lumen of the cannula during implantation, as well as minimize tissue damage at the target location.

The invention may include and one of the embodiments described above or any combination thereof.

### BRIEF DESCRIPTION OF THE DRAWINGS

FIGS. 1A-C are of embodiments of an injection device. FIG. 1A is a longitudinal cross-section of the distal end of the injection device having an implant loaded in the cannula lumen; FIG. 1B is a longitudinal view of the distal end of an injection device; FIG. 1C is a longitudinal view of the distal end of an injection device.

FIG. 2A is a longitudinal view of one embodiment of an injection device; FIG. 2B is an inset of the injection device distal end.

FIG. 3A is a longitudinal view of one embodiment of an injection device; FIG. 3B is a cross-sectional view of the distal end of the injection device having a detent; FIG. 3C is a longitudinal view of one embodiment of an implant; FIG. 3D is a front view of one embodiment of an implant

FIG. 4A is a longitudinal view and cross-section of the distal end of one embodiment of an injection device having an implant loaded in the lumen; FIG. 4B is a longitudinal view of one embodiment of a probe for use in an injection device; FIG. 4C is an inset of a probe distal end tab configuration; FIG. 4D is a side view of one embodiment of an implant; FIGS. 4E & F are cross-sectional views of probe/implant configurations.

FIG. 5A is a longitudinal cross-section of one embodiment of an injection device with a handle configuration in a first position; FIG. 5B is a longitudinal cross-section of an injection device with a handle configuration in a second position.

FIG. 6A is a longitudinal view of one embodiment of an injection device having a configured cannula and probe handle arrangement. FIG. 6B is a longitudinal view of one embodiment of a probe having a configured probe handle.

FIG. 7 is a longitudinal cross-section of an embodiment of a portion of an injection device.

FIG. 8 is a longitudinal view of one embodiment of an implant.

FIG. 9A is a longitudinal view and cross-section of an embodiment of an injection device; FIG. 9B is an inset of a cross-sectional view of the injection device including an implant and a probe configured for use with the injection device; FIG. 9C is an inset of a perspective view of part of an implant configured use with a probe of an injection device.

FIG. 10A is a longitudinal cross-section of an embodiment of an injection device; FIG. 10B is a longitudinal cross-section of an embodiment of a probe for use in an injection device; FIG. 10C is a longitudinal view of an embodiment of an injection device; FIG. 10D is an inset of a cross-sectional view of an injection device; FIG. 10E is an inset of a cross-sectional view of an injection device; FIG. 10F is a longitudinal view of the distal end of one embodiment of an injection device.

FIGS. 11A-C are longitudinal cross-sections of an embodiment of an injection device used to deliver an implant loaded therein shown in various positions during use.

FIGS. 12A-C are longitudinal cross-sections of an embodiment of an injection device used to deliver an implant loaded therein shown in various positions during use.

FIG. 13 is a flow diagram of one method for positioning an implant using an injection device.

FIG. 14 is a flow diagram of one method for positioning an implant using an injection device.

FIG. 15 is a flow diagram of one method for positioning an implant using an injection device.

FIG. 16 is a flow diagram of one method for loading an implant in an injection device using an injection device.

### DETAILED DESCRIPTION OF THE PREFERRED EMBODIMENTS

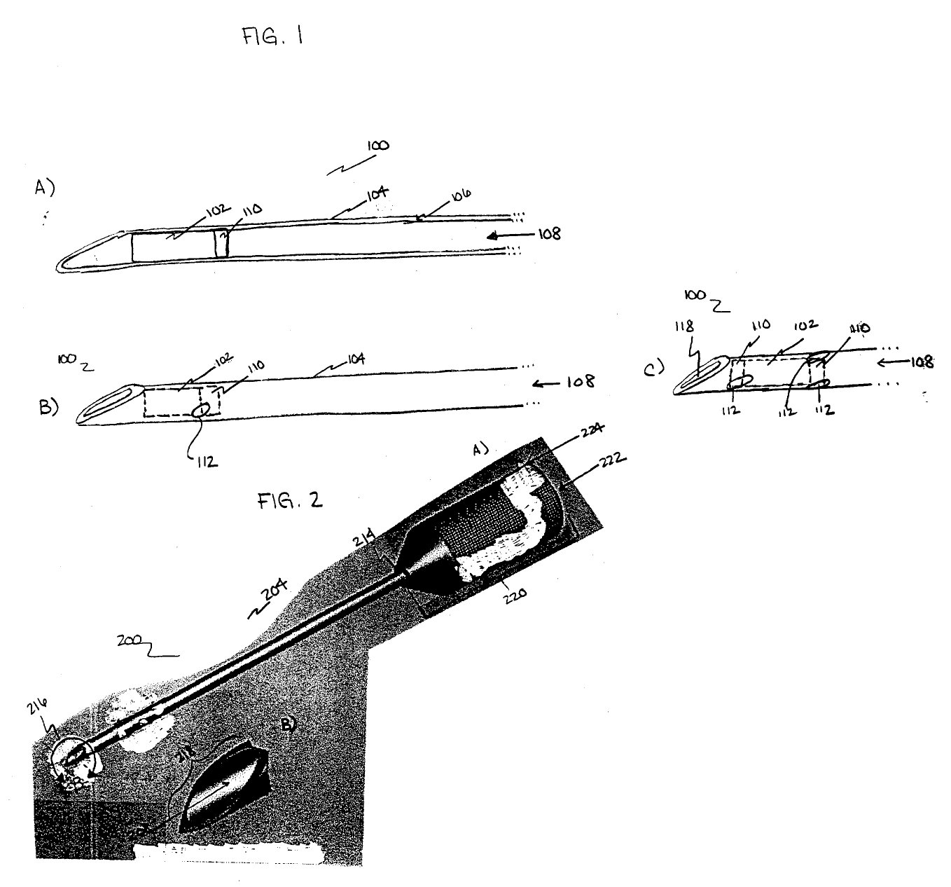

FIGS. 1A-C are of embodiments of an injection device 100 for positioning an implant 102 in the body. The injection device 100 may include a cannula 104 having a substantially cylindrical cannula wall 106 forming a cannula lumen 108. An implant 102 may be configured for positioning within the cannula lumen 108 and the implant 102 may have at least one external electrode 110 (FIG. 1A). Further, at least one fluid communication channel 112 (“channel”) may be formed in the cannula wall 106 to permit interstitial fluid from the target location to enter into the cannula lumen 108 and contact the implant 102 (FIG. 1B). The channel 112 may be formed at a location along the cannula length, such that the channel 112 is substantially aligned with the implant external electrode 110.

In one embodiment, the cannula wall 106 may have a plurality of channels 112 formed therein. Where a plurality channels 112 may be used, the channels 112 are spaced longitudinally or axially, or spatially offset so as to maximize the structural integrity of the cannula wall 106. In yet another embodiment, the implant may include two external electrodes 108 (FIG. 1C). In that embodiment, the channels 112 may be positioned longitudinally, such that at least one channels 112 is substantially aligned with each electrode 108. The cannula distal opening 118 may also serve as a communication channel permitting fluid from the target location to enter the cannula lumen 108.

FIG. 2 is a depiction of one embodiment of an injection device 200 which may be used in this invention. In this embodiment, a cannula 204 has a cannula proximal end 214 and a cannula distal end 216. The cannula distal end terminates in a cannula distal opening 218 which may be a blunt end, a beveled end or a double beveled end, for example. The cannula proximal end 214 may be integrally formed into or attached to a separately formed cannula handle 220. The cannula handle 220 may have formed therein a cannula handle lumen 222, wherein the cannula lumen 208 and cannula handle lumen 222 are continuous. The cannula handle outer surface 234 may be configured with a textured surface, such as ridges or cross-hatching to facilitate the user's grip on the cannula handle during use. The cannula handle 220 may also be configured for interaction with a probe. A variety of interactive handle mechanisms will be described below.

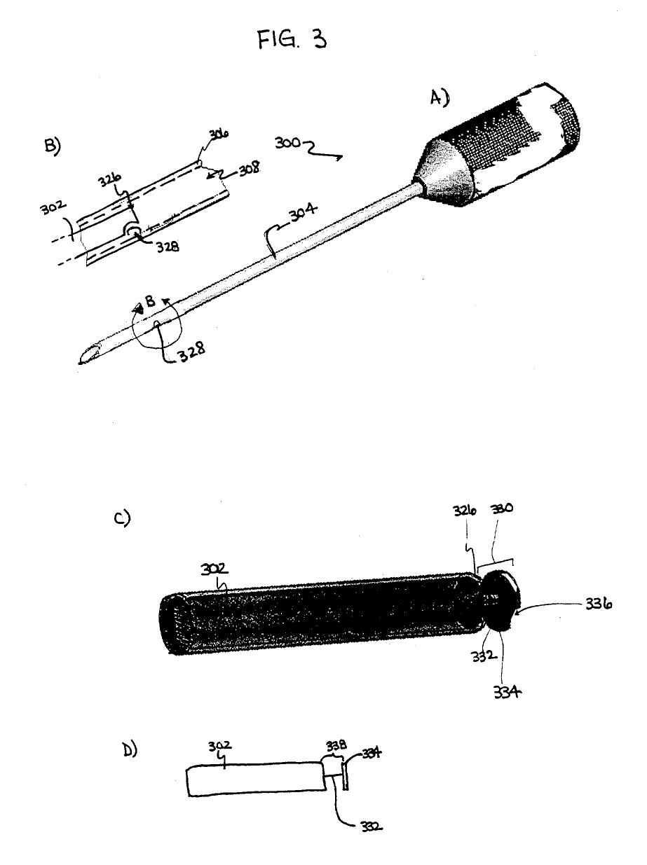

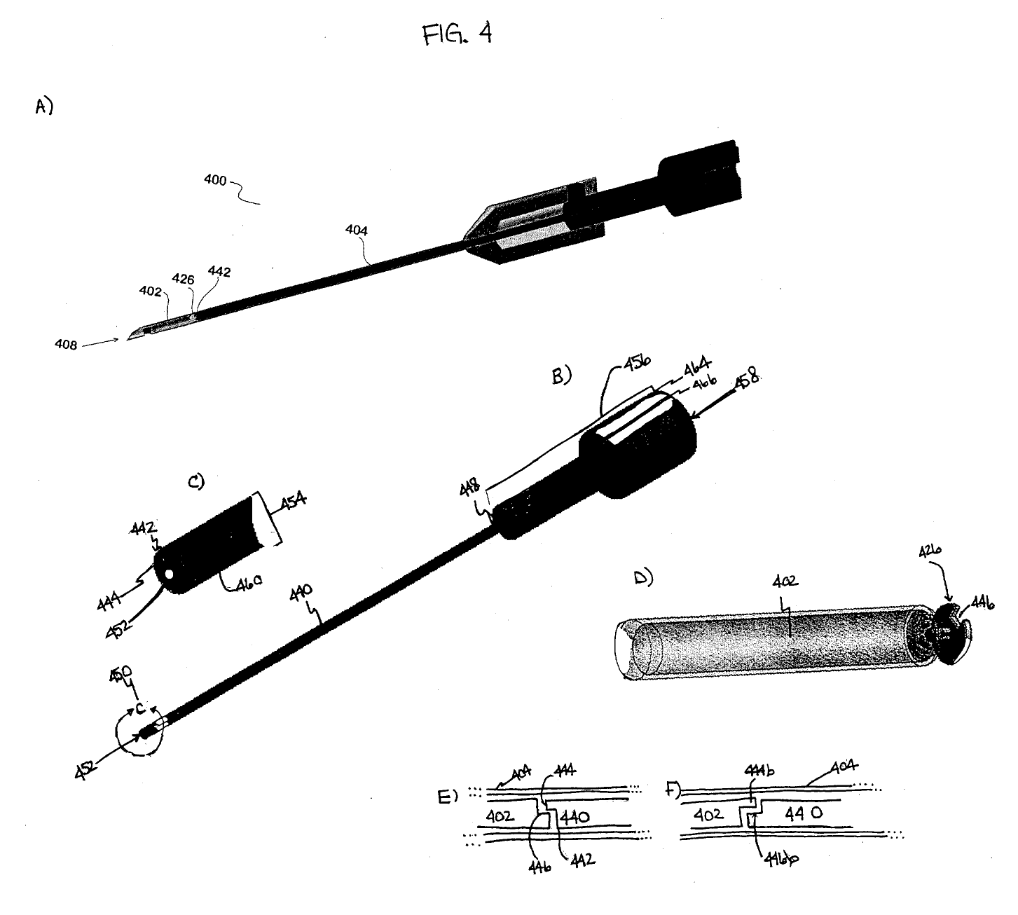

As shown in FIG. 3A, in one embodiment, the injection device 300 may include a cannula 304 and an implant 302 positioned within the cannula lumen 308, such that an implant end surface 326 is configured to releasably engage a surface within the cannula lumen 308. As depicted in FIG. 3B, in one embodiment, the cannula lumen 308 may be modified to include a detent 328. The detent 328 may be integral to the cannula wall 306 or may be formed as a separate structure which is then attached to the cannula lumen 308. Some methods of constructing the detent 328 include, but are not limited to, injecting a bump of extrinsic material, bending in a tag of the cannula wall material, and inserting a pin/peg of extrinsic material through a slot in the cannula wall. The detent 328 may be formed in any shape having a detent cross-section.

Further, an implant surface 326 may be modified to form a retaining member 330 (FIGS. 3C & D). The retaining member 330 may be integral to the implant 302 or may be formed as a separate structure which is then attached to the implant surface 326. In one embodiment, the retaining member 330 may include a post 332 and an annular ring 334, having a notch 336 therein (FIG. 3C). The post 332 length may be selected such that the detent 328 fits within a detent space 338 formed between the implant surface 326 and the annular ring 334. The notch 336 in the annular ring 334 may be formed in any shape having notch cross-section that is compatible with the detent cross section, such that the notch 336 can move slidably past the detent 328 when the detent 338 and notch 336 are axially aligned. Further, the detent may be constructed such that the detent can be retracted from the cannula lumen when it becomes desirable to release the implant.

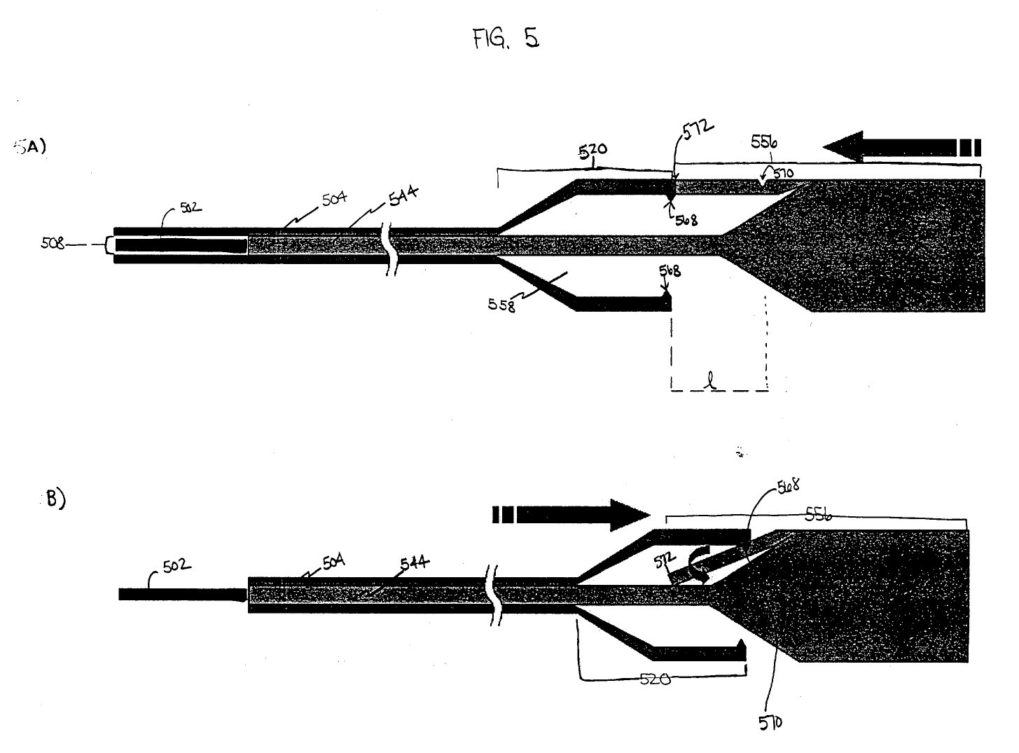

As depicted in FIG. 4A, in one embodiment the injection device 400 may include a cannula 404, an implant 402 positioned in the cannula lumen 408, and a probe 440 positioned such that an implant end surface abuts the probe distal end surface 442. Both the implant end surface 426 and probe distal end surface 442 may be configured to prevent the implant 402 from rotating with respect to the probe 440 while the surfaces abut. In one embodiment, the configurations which prevent the implant from rotating with respect to the probe 440 while the implant end surface 426 abuts the probe distal end surface 442 may also permit the implant 402 to separate longitudinally from the probe 440 during implantation. FIG. 4B depicts one embodiment in which the probe distal end surface is configured as a tab 444 having a cross-sectional shape, such as a rectangular tab 444 (FIG. 4C). The tab 444 may be formed integrally in the probe 440 or may be formed as a separate structure which is attached to the probe distal end surface 442.

Further, the implant end surface 426 may be configured as a slot 446 having a cross-sectional shape selected to be compatible with the tab cross-sectional shape, such as a rectangular slot 446 (FIGS. 4D & E). The slot 446 may be formed integrally in the implant 402 or may be formed in a separate structure which is attached to the implant end surface 426.

In an alternative embodiment, the slot 446b may be formed on the probe distal end surface 442 and the tab 444b on the implant end surface 426 (FIG. 4F). The tab and slot cross-sectional shapes may be selected such that the tab/slot maintain a fixed orientation relative to one another. However, the tab and slot cross-sectional shapes should also be selected such that once the implant is positioned in a target location, the probe can be separated from the implant without substantially modifying the implant's longitudinal or axial orientation.

FIG. 4B is a depiction of one embodiment of a probe 440 which may be used in this invention. In this embodiment, the probe 440 has a probe proximal end 448 and a probe distal end 450. The probe distal end 450 may be modified for interaction with an implant 402, as described above. The probe may have a probe lumen 452 extending from the probe distal end 450 to the probe proximal end 448. The probe outer diameter 454 may be such that the probe outer diameter 454 moves within the cannula lumen 408 with minimal friction, but also minimal horizontal or vertical movement. The probe proximal end 448 may be integrally formed into or attached to a separately formed probe handle 456. The probe handle 456 may have a probe handle lumen 458, the probe lumen 452 and probe handle lumen 458 may be continuous. The lumens may be centrally located within the probe 440, and probe handle 456 respectively. In the alternative, a probe groove 460 may be formed along one side of the probe 440 from the probe distal end 450 to the probe proximal end 448 which communicates with a probe handle lumen 458. The probe handle lumen 458 may terminate in a syringe port 460 (not shown) configured to receive any standard syringe. The syringe port may permit drawing back during the procedure to assess for bleeding or withdrawal of any material from the target site, or permit the concurrent delivery of agents to the target location, as described below.

The probe handle outer surface 464 may be configured with a textured surface, such as ridges or cross-hatching to facilitate the user's grip on the probe handle 456 during use. The probe handle 456 may also be configured to include a marker 466, wherein the position of the marker 466 on the probe handle 456 is in a fixed axial orientation relative to the probe handle outer surface 464 as the tab 444 or slot 446b modification on the probe distal end 450. The marker 464 may be formed in the probe handle outer surface 464 as an indentation or may be formed of a separate component added to the probe handle 456, for example.

Further, in some embodiments, the probe groove 460 cross-sectional shape may be selected such that the probe groove 460 moves slidable along a detent 428 within the cannula lumen 408 when the cannula 404 is axially aligned to permit longitudinal movement relative to the probe 440.

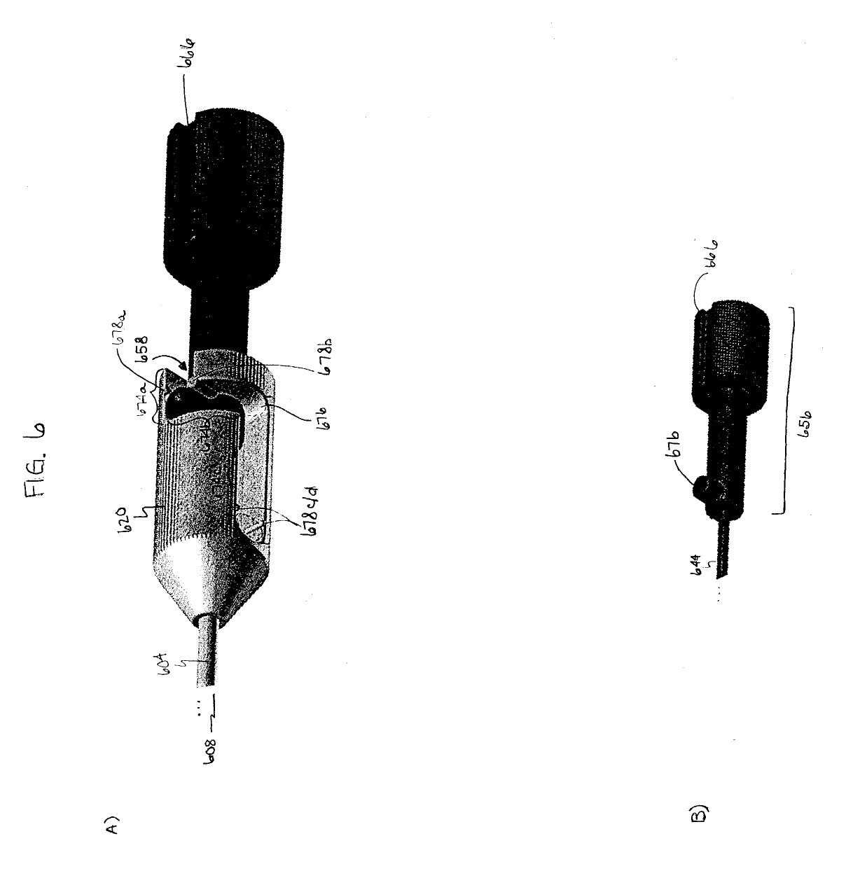

Further, in some embodiments, the cannula handle and a probe handle may be configured to permit defined longitudinal and/or axial movement relative to one another during the implantation process. These embodiments are advantageous at least in maintaining the orientation of an implant at the target location during implantation. FIGS. 5A & B depicts one embodiment of a handle configuration for permitting defined longitudinal movement of a cannula 504 and probe 544 relative to one another. In this embodiment, the probe 544 and probe handle 556 are configured such that they remain stationary while the cannula 504 and cannula handle 520 slide longitudinally over the probe 544. Further, the cannula handle 520 may include a discrete pin or ridge 568 which extends within the cannula handle lumen 558. Correspondingly, the probe handle 556 may include a discrete hole or trough 570 which extends into the probe handle 556. The distance from the distal most end of the probe handle to the hole/trough 570, “I”, may be selected to be substantially the same as the length of the implant 502.

In this embodiment, when the cannula 504 is moved proximally relative to a stable probe 544, the peg 568 moves longitudinally relative to the hole 570, until the pin 568 comes to rest in the hole 570. Therefore, the cross-sectional shape of the pin 568 and hole 570 may be selected such that the peg fits within the hole. Further, the proximal, longitudinal movement of the cannula 504 for a distance, I, may be sufficient to expose the implant 502 from within the cannula lumen 508 to the target tissue.

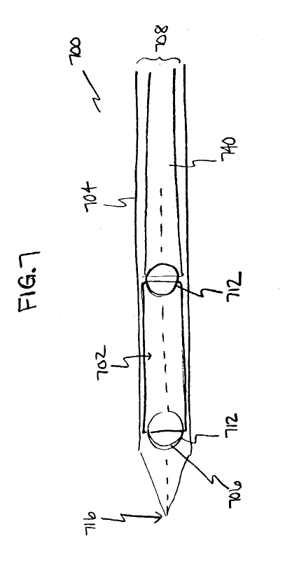

FIG. 6A depicts one embodiment of a handle configuration for permitting defined longitudinal and axial movement of a cannula 604 and probe 644 relative to one another. In this embodiment, the cannula 604 and cannula handle 620 are configured such that they slide longitudinally over the probe 644 and probe handle 656. Further, the cannula handle 620 may include a track 674 having a track proximal section 674a, track axial section 674b and a track distal section 674c extending through the cannula handle 620. In this embodiment, the cannula 604 and cannula handle 620 are configured such that they slide longitudinally over the probe 644 and probe handle 656. The track 674 may further be configured such that locking detents 678 are located in select positions within the track 674, such that greater force must be exterted between the probe handle 656 and the cannula handle 620 as they are moved relative to one another. For example, locking detents 678a/b may be positioned in the track proximal section 674a, such that the probe peg 676 holds the probe handle 656 in a first position relative to the cannula handle 620 after the application of longitudinal force to move the cannula handle 620 relative to the probe handle 656. With the application of sufficient axial rotational force, the cannula 604 and cannula handle 620 may move past the locking detent 678b around peg 676 into a second position in the track axial portion 674b. Finally, locking detents 678c/d may be located at the distal end of the track distal portion 674c, such that with sufficient force, the cannula handle 620 is moved into a third, locked position relative to the probe handle. The proximal, longitudinal movement of the cannula handle 604 for a distance, about equal to the distance of the track distal portion 674c, may be sufficient to expose the implant from within the cannula lumen 608 to the target tissue. Further, the axial movement of the cannula handle 604 for a distance about equal to the track axial section 604b, may be sufficient to align the implant to releasably disengage from a configured surface in the cannula lumen 608. Also, the positioning of the probe handle marker and/or peg 676 may be selected to represent the orientation of an implant 602 (not shown) in the target location. Where the probe 644 is maintained in a stable position relative to a moving cannula 604, an implant may be maintained at a stable longitudinal position during withdrawal of the cannula 604. Where the probe distal end has been configured to prevent the implant from rotating relative to the probe 644, an implant will be maintained at a stable axial position during withdrawal of the cannula 604.

More particularly, in use an implant may delivered within an injection device utilizing this handle configuration. After overcoming an initial locking resistance due to locking detents the cannula is rotated through 90° along its path over the probe's peg, while the probe and implant is held stationary via the probe's handle. A cannula detent thus comes to align itself with an implant notch and corresponding probe travel groove, so freeing the implant. Continuing the cannula along a longitudinal path by withdrawing it for the length of the implant within, the implant becomes exposed to the target location and is to be held by the friction contact of the surrounding tissues. Finally the cannula locks over the probe's peg at the end of its travel course.

In some clinical situations, concerns exist regarding the use of beveled needles in areas where the arteries and nerves themselves may often be narrower than the needle. This is because the beveled edge of the needle may cut nerves and other tissues when the needle is moved through the tissue. Thus to minimize the trauma associated with a beveled instrument, while still achieving all the goals listed previously, alternative embodiments are described here.

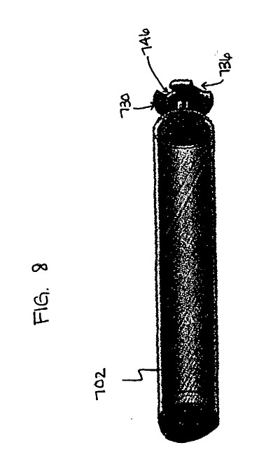

As depicted in FIG. 7, in one embodiment of the invention an injection device 700 may include a cannula 704 having a distal end formed into a trochar 716 and an implant 702 releasably engaged within the cannula 704. In an alternative embodiment, the injection device 700 may further include a probe 740 to facilitate the release of an implant 702 placed within the cannula 704 into the body at the target location. In these embodiments, the trochar-tipped cannula 704 may be constructed such that the cannula 704 separates longitudinally to deliver the implant 702 at the target location.

The trochar-tipped cannula 704 may further include modifications, such as those described above to accomplish the objectives of this invention. For example, the trochar-tipped cannula 704 may include channels 712 in the cannula wall 706 to facilitate fluid communication with the implant 702. Also, the cannula lumen 708 may include detents 728 to longitudinally orient the implant 702 in the cannula 704. Also, the cannula or probe 740 may be configured to axially orient the implant 702 in the cannula 704. The implant's axial alignment may be controlled by a suitable male-female interlocking arrangement, between the cannula wall and the implant or one of the electrodes, for example.

In one embodiment, the invention may include an implant configured to be injected by an injection device into body tissue or a body cavity and configured with a surface that interlocks with a surface in the injection device. This embodiment may be used to restrict axial rotation or longitudinal movement of the implant relative to the injection device during implantation.

In one embodiment, the implant may be configured to maintain longitudinal alignment between the implant and the injection device while the implant is within the injection device and during implantation. As described above, FIG. 3B depicts one example of a modified implant.

In one embodiment, the implant may be configured to have an interlocking surface to maintain axial alignment between the implant and the injection device while the implant is within the injection device and during implantation. Further, in one embodiment, the interlocking surface is configured to allow the implant to be separated longitudinally from the injection device during implantation. As described above FIG. 4C is one example of a modified implant.

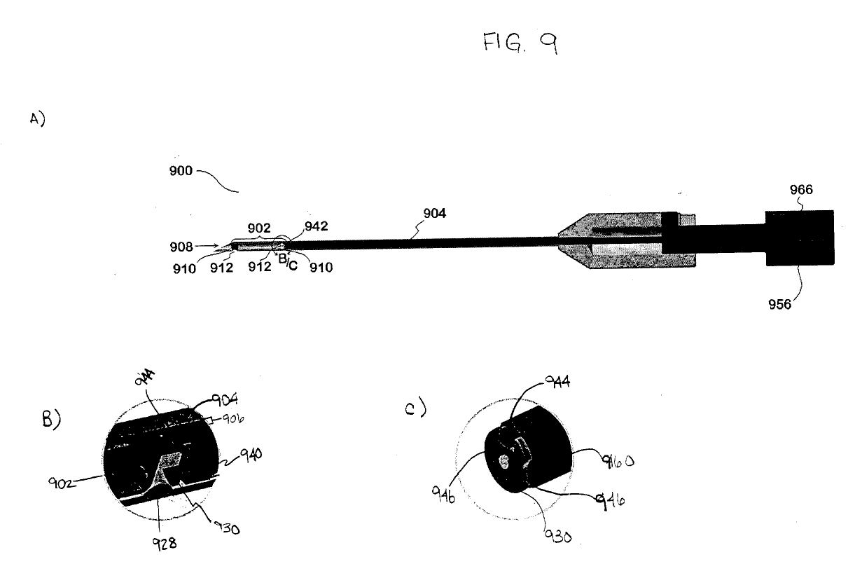

Further, in some embodiments, the implant may be configured to maintain both the longitudinal and axial position of the implant relative to the injection device. One example of an implant according to this embodiment is depicted in FIG. 8. In this example, the implant 802 may have a retaining member having both a slot 746 and notch 736 therein for interaction with a probe tab and cannula lumen detent, respectively.

In one embodiment, the implant may be modified such that a slot and notch are located at different locations on the implant itself or by way of structures attached to the implant.

One example of an implant which may be useful in this invention is the BION™ (BIONic Neurons; Alfred E. Mann Institute, University of Southern California). BIONs™ are a new class of implantable medical device: separately addressable (up to 256), single channel, electronic microstimulators (16 mm long×2 mm in diameter), that can be injected in or near muscles and nerves to treat paralysis, spasticity and other neurological dysfunctions. A BION typically may include a tantalum electrode at one end and an iridium electrode at the opposite end. Each BION™ may receive power and digital command data by a radio frequency electromagnetic field to produce functional or therapeutic electrical stimulation. A BION typically may include a tantalum electrode at one end and an iridium electrode at the opposite end. For use in this invention, the electrodes may be configured for selective interaction with the surfaces of an injection device, including but not limited to the cannula lumen or probe distal end for example.

In order to produce functionally useful reanimation of a paralyzed limb, it may be desirable to provide sensory feedback about the posture and motion of the limb in order to control the details of muscle activation achieved by electrical stimulation. Various types of sensors may be incorporated into implants such as BIONs to detect such posture and motion. The data provided by these sensors can be telemetered out to a control system by electromagnetic signaling. One useful sensing function may consist of inferring the relative distance and orientation between a pair of implants located in muscles by measuring the strength of electrical or magnetic coupling between them. As the posture of a joint changes, the length and position of muscles acting across that joint may also change, carrying the implants with them.

Another useful sensing function may be accomplished using an accelerometer, which may be sensitive to both the induced motion of the limb in an inertial frame of reference and the steady pull of gravity in one direction in that inertial frame of reference. In both of these sensing modalities, it is important to control the position and orientation of the implants in the body, which is an objective of the subject invention. In the case of a BION implant containing a one- or two-axis accelerometer, axial rotation of the cylindrical implant may substantially change the sensitivity of the accelerometers in the normal body posture, making it important to control the orientation of the implant in this axis during the implantation process.

In yet another sensor, the bioelectrical fields generated by an electrically active tissue such as muscle or nerve may be detected by implant electrodes, depending on the orientation of those electrodes with respect to the bioelectric source. Loeb, et al., “Bion System for Distributed Neural Prosthetic Interfaces,” Joumal of Medical Engineering and Physics, 23: 9-18, 2001.

Other types of implants which may be positioned with high precision could also be utilized in this invention including, but not limited to, other miniaturized electrical devices and/or mechanical devices (e.g., nano-devices, micromachines, microstimulators), implants containing various bioactive agents (like chemo-therapeutic agents, radiotherapeutic beads), tissue cultures or cell cultures.

In one embodiment, the implant comprises a delivery capsule including cargo to be delivered to the target location. In some embodiments, the capsule may be permeable to cargo, such that the cargo diffuses from the capsule and into the target location when implanted. In some embodiments, the capsule may be dissolvable so as to release the cargo at the target site when implanted. In one embodiment, a dissolvable capsule may be constructed of materials including, but not limited to polyglactic acid or polydioxanone, or a combination of polyglactic acid or polydioxanone.

A variety of implant shapes and sizes of implants utilized according to this invention are envisioned by modification of the implant and/or injection device accordingly. Where the implant is a device, the implant itself may be modified in configuration to accomplish the objectives of this invention. Alternatively, where the implant is a capsule, the capsule may be configured to accomplish the objectives of this invention without modification to the cargo.

In alternative embodiments, the injection device is constructed of materials so as to be compatible with the implant being injected. In some embodiments, it is desirable to select materials which do not interfere with the ability to test the effectiveness of the implant at the target location, prior to releasing the implant from the injection device. For an implant that receives power and/or command signals by electromagnetic transmission, it may be important that the materials of the injection device not interfere with these transmissions by electrically shielding or deflecting electromagnetic fields. For example, electrically conductive material surrounding or adjacent to an implant may support eddy currents that dissipate the electromagnetic radiation, preventing it from reaching the implant.

In one embodiment, the injection device may include a cannula including materials that will not shield/interfere with electromagnetic signal configured to contain the implant while the injection device is being inserted into the body. In one embodiment of the invention this cannula, made of a material that will not shield/interfere with electromagnetic signals, is used for the insertion and pre-testing of an implant which communicates using electromagnetic radiation. Materials useful for this embodiment, include, but are not limited to plastic, ceramic, glass or any combination thereof.

In an alternative embodiment, the injection device may include a cannula including electrically insulating material that is configured to contain the implant while the injection device is being inserted into the body. In one embodiment of the invention this electrically insulating cannula is used for the insertion and pre-testing of an implant which communicates using electricity. The material used for the housing of electrically insulating material may provides a degree of insulation which is at least one order of magnitude or ten-times greater that the body fluids expected to be in contact with the housing and implant. The material's resistivity may be selected to be at least greater than that of body tissues (±10² Ω.cm). Materials useful for this embodiment, include, but are not limited to plastic, ceramic, glass or any combination thereof. This embodiment may be useful where the implant is a BION™, and where pre-testing occurs before the BION™ is released from the injection device, and where the BION utilizes the transmission of electrical impulses to a test position in the body.

Further, materials used for the embodiments of the injection devices are may be selected so as to ensure that the injection device is sufficiently rigid and the distal tip can be made sharp enough to be inserted at the entry site. Further, the materials may be selected so that the injection device is sufficiently pliable to be manipulated by the user without breaking. By way of example, the materials selected may exhibit rigidity and pliability characteristics similar to a 17 gauge stainless steel needle, and for some embodiments, stainless steel may be selected as the material. Materials may be selected so as to withstand lateral forces equivalent to the approximately 96-424 g exerted upon a 12 gauge needle during implantation through soft tissue. By way of example, a 12 gauge plastic cannula having a wall-thickness of 0.0125″for a material with a flexural modulus of 17,900 MPa has been determined to have similar flexural strength to a standard 17 gauge stainless steel needle. In some embodiments, it may be desirable to increase the stiffness of a polymeric material by longitudinal fiber filling (for example with carbon or glass). The material selected may be impact resistant and sterilizable by some means (e.g. a softening temperature>125° C. for autoclaving).

Materials used for all parts of the instrument, may be selected so as to be are biocompatible, sterilizable, suited to required manufacturing dimensions and tolerances, machineable to incorporate required features (e.g., predictable forces at points of locking between parts), able to be fused with one another where required (e.g., the cannula with the cannula handle), and able to move relative to one another as required.

Examples of materials which may be useful in this invention include, but are not limited to VECTRA B130 (30% glass-filled Liquid Crystal Polymer, Ticona); STAT-KON RC (30% carbon-filled Polyamide 66, LNP); VERTON RF-700-12 (60% glass-filled Nylon 6/6, LNP); and RYNITE 555 (55% glass-filled Thermoplastic Polyester Resin, DuPont).

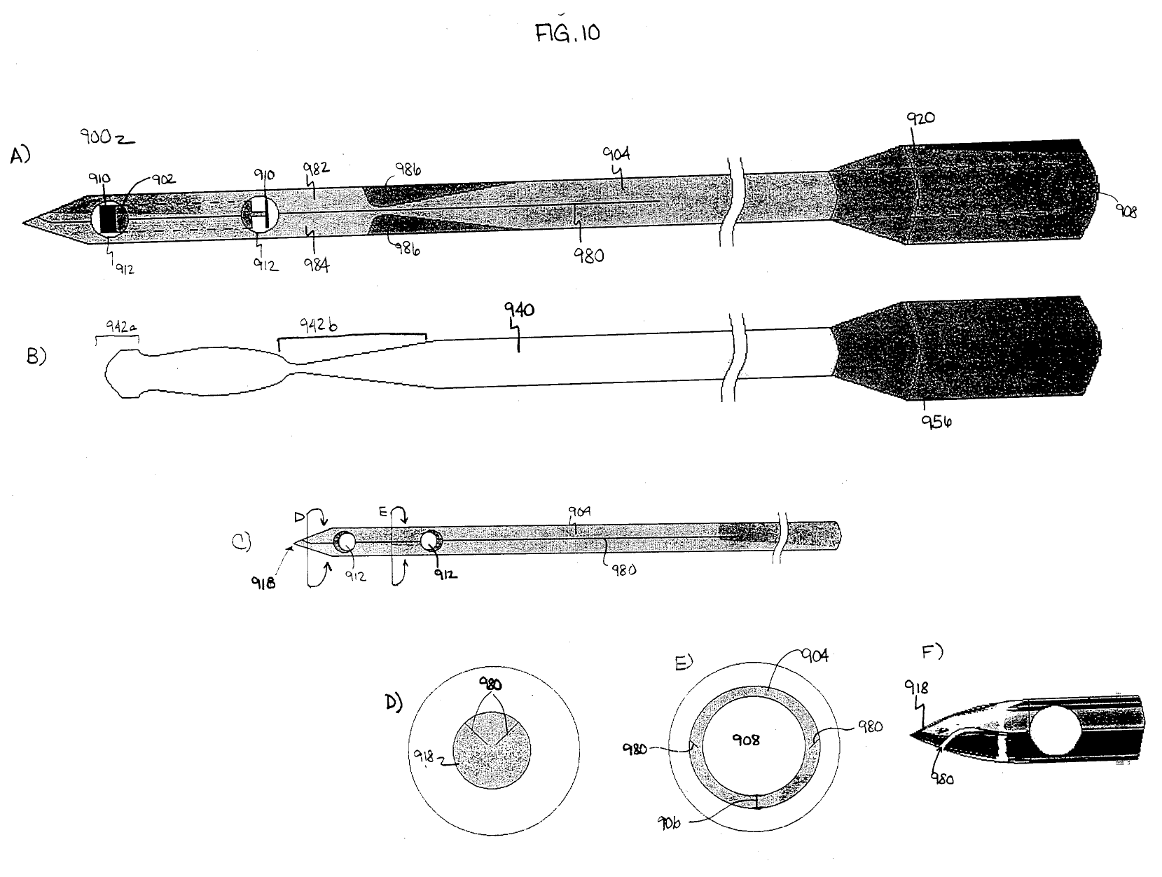

One example embodiment is depicted in FIG. 9. In this embodiment, the injection device comprises cannula having a cannula lumen, a probe having a distal end within the cannula lumen; and an implant having an implant end within the cannula lumen. Further, the cannula may include a detent that protrudes inwardly into the lumen and the implant may include an annular ring on the surface that is engaged with the tab. Further, a notch in the annular ring on the implant which is larger than, but aligns with the detent when the cannula is rotated axially with respect to the implant. Finally, an implant end surface is engaged with a probe distal end surface such that rotational, but not longitudinal movement between the probe and implant is prevented while the surfaces are engaged.

FIG. 9A depicts one example of an injection device 900 of the present for use with an implant, such as a BION™ 902. The components of the injection device 900 are designed to fit together as follows: the BION™ 902 is loaded inside the distal end of the cannula lumen 908 and abutting the probe distal end 942. As shown in FIG. 9B, the BION™ 902 is retained in a longitudinal position by the detent 928 distal to of the BION's™ 902 Iridium electrode 930, and the probe 940 proximal to this electrode. As shown in FIG. 9C, the BION's™ 902 axial orientation is maintained by the probe tab 944 which fits into the slot 946 in the Iridium electrode 930. The tab/slot 944/946 arrangement is aligned with a longitudinal marker groove 966 in the probe handle 956, so that the clinician is able to axially orient the BION™ as desired at insertion. The detent 928 is constructed to be slidable in the notch 446 and probe groove 460. The cannula may include a plurality of channels 912 spaced so as to be in the vicinity of the BION's™ iridium and tantalium external electrodes 910/930.

In this example, channels 912 in the cannula wall 906 are positioned adjacent to the BIONs™ electrodes 910, and together with the cannula distal opening 918, provide electrical access to the tissues at the target position. These channels 912 facilitate repeated stimulation by the implant 902 at any point while traversing the tissue path so as to determine target location, and help avoid damage to any nerves. Further, these channels 912 also enable optimal implant positioning by stimulating the target with the BION™ itself; using a specific antenna-BION™ couple destined for use with that patient. The proximal pair of channels 912 depicted are not directly opposite one another, but rather are designed with a slight offset, so as to maximize the cannula wall surface area and hence strength in this area, whilst still adequately exposing the BIONs™ Iridium electrode to the body fluids. Similarly, the distal most channel 912 is unpaired, once again to maximize the cannula wall's 906 surface area and hence strength in this area, and together with the cannula distal opening 918, adequately exposing the BION's™ Tantalum electrode to the body fluids.

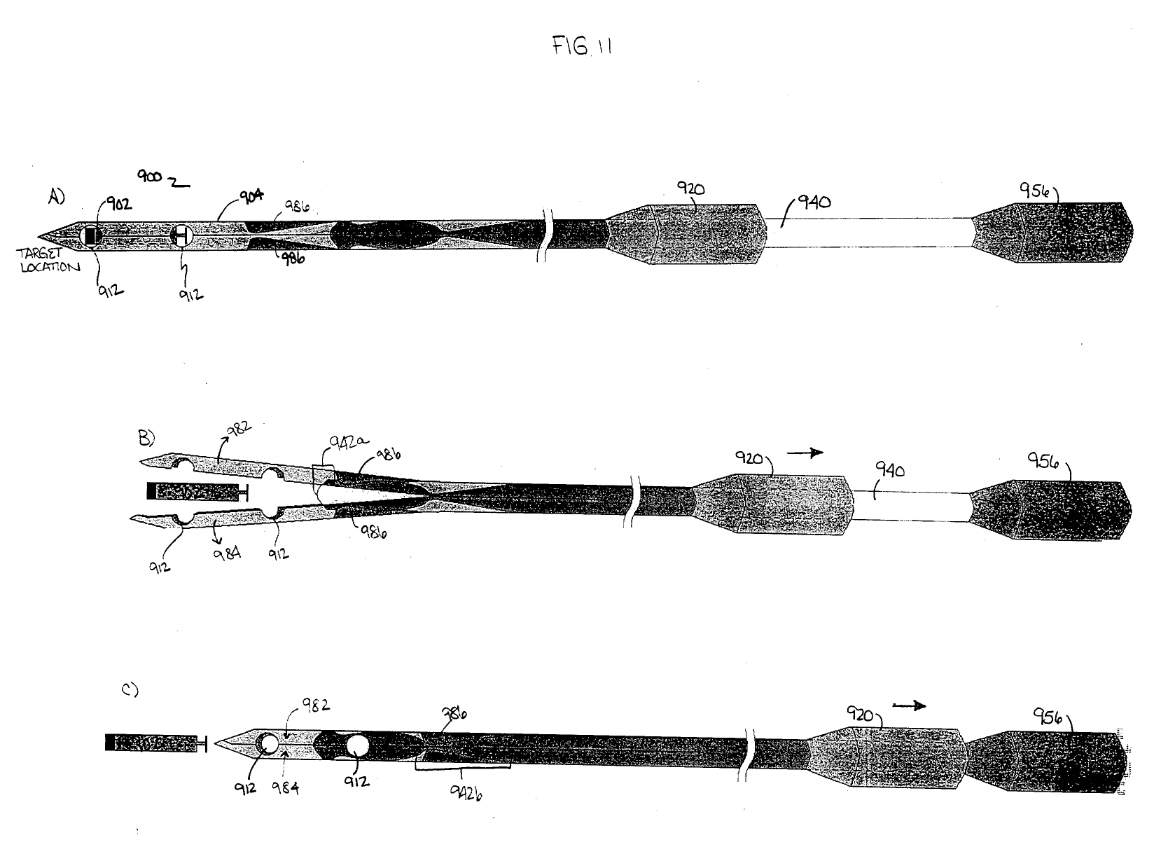

Another example embodiment of an injection device is depicted in FIG. 10A. In one embodiment, the injection device 900 may include a cannula 904 having a slit 980 in the distal portion of the cannula wall 906 (to create a cannula upper casing 982 and a cannula lower casing 984) (FIG. 10B). To avoid movement of the cannula upper and lower casings 982/984 relative to one another under tension, the slit 980 may be diagonalized in section and/or curved at the cannula distal end 918 (FIGS. 10D, E). Alternatively, the slit 980 may be only partial, such that the slit 980 does not extend though the entire thickness of the cannula wall 906. Alternatively, a protruding ridge running longitudinally along one of the slit edges may be configured so as to fit into a corresponding groove running longitudinally along the other of the slit edges. Finally, the slit 980 in the cannula distal tip 918 may be curved downward so as to minimize separation of the two cannula portions during insertion (FIG. 10F). As described above, the cannula 904 may have a plurality of channels 912 aligned with the implant 902 (FIGS. 10A & C).

The cannula lumen 908 and the probe 940 (FIG. 10B) may be modified in shape, such that the movement of the cannula 904 relative to the probe 940 results in the opening of the upper and lower casings 982/984 relative to one another to release the implant 902. For example, the cannula lumen may include one or more release detents 986, and the probe distal end portion 942a configured so as to have a diameter less than that of the unmodified cannula lumen 908, but greater than the diameter of the lumen 908 as modified with release detent(s) 986, and a probe distal portion 942b configured to a have a cross-section compatible with the cross-section of the modified lumen (FIG. 10B).

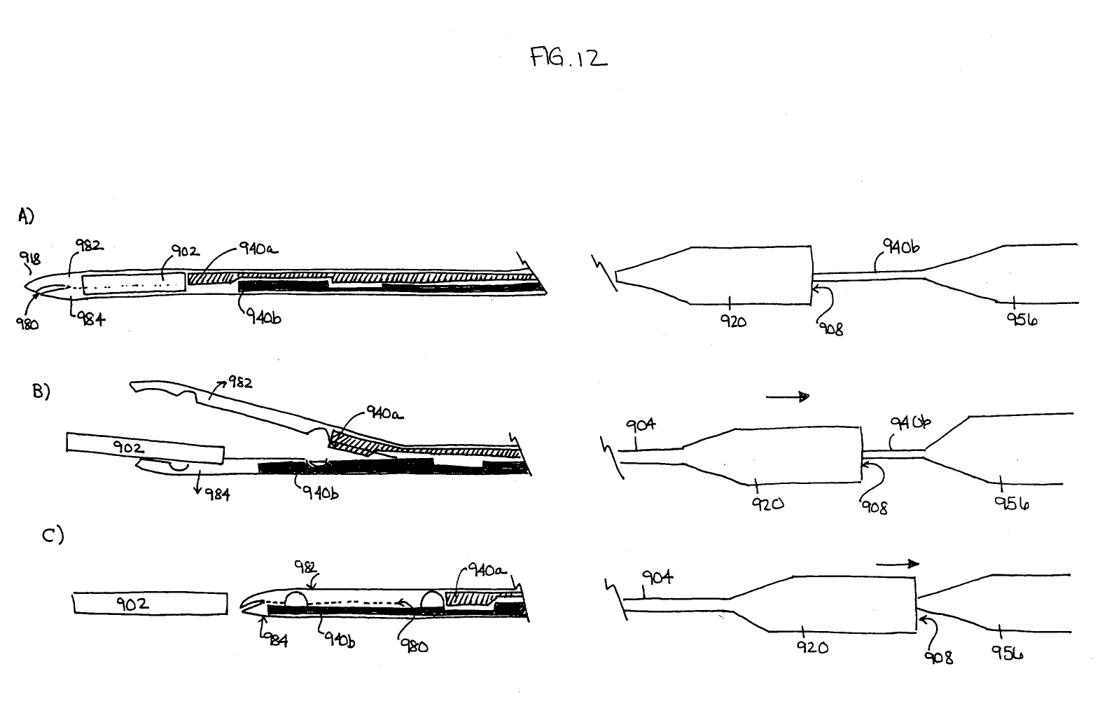

FIGS. 11A-C depict the use of this injection device 900 to position and release an implant 902 at a precise longitudinal location. First, the injection device 900 having an implant 902 therein is directed into a target location, and the cannula 904 is stabilized relative to the target location (FIG. 11A). Next, the cannula 904 is moved proximally relative to the probe 940 to a first position, wherein the probe distal end portion 942a contacts the releasing detent(s) 986. Due to the displacement pressure created in the cannula lumen 908, the upper and lower casings 982/984 move away from one another, and the cannula opens at the slit 980. The opening motion of the cannula permits the implant 902 to be released into the target tissue. Finally, the cannula 904 is moved again proximally relative to the probe 940 to a second position, wherein the probe distal portion 942b comes into alignment with the releasing detent(s) 986. Due to the fit between the cross-section of the detent(s) 986 and the probe distal portion 942b, the upper and lower casings 982/984 move together, and the cannula 904 closes, behind the implant. The injection device 900 can then be removed from the patient as a single unit.

Another example embodiment of an injection device is depicted in FIG. 12. In one embodiment, the injection device 900 may include a cannula 904 having a slit 980 in the distal portion of the cannula wall 906 (to create a cannula upper casing 982 and a cannula lower casing 984). The probe 940 may include a probe upper unit 940a and probe lower unit 940b which are inserted into the cannula lumen 908 behind the implant 902. The probe upper unit 940a may be attached to the cannula lumen 908, such that there is minimal or no relative movement between them. The probe lower unit 940b and implant 902 are thus held in a stable arrangement within the cannula lumen 908 and probe upper unit 940a combined unit by this relationship.

As demonstrated in FIG. 12A, after the implant is positioned in the target location by the injection device, a user may first hold the probe lower unit 940b stationary and slide the cannula 904/probe upper unit 940a proximally to a first position. In doing so, the cannula upper and lower casings 982/984 will be opened in the region of the slit by the camming action of the upper probe unit 940a over the lower probe unit 940b (FIG. 12B). The implant 902 will be released from the cannula lumen 908, being held by the friction of contact with the surrounding tissues as the cannula 904/probe upper unit 940a moves proximally. As the motion of moving the cannula 904/probe upper unit 940a proximally continues, the probe upper unit 940a will move into a second position relative to the probe lower unit 940b, thus allowing the cannula upper and lower casings 982/984 to close behind the released implant 902 (FIG. 12C). Again, the injection tool can be withdrawn from the patient as a single unit.

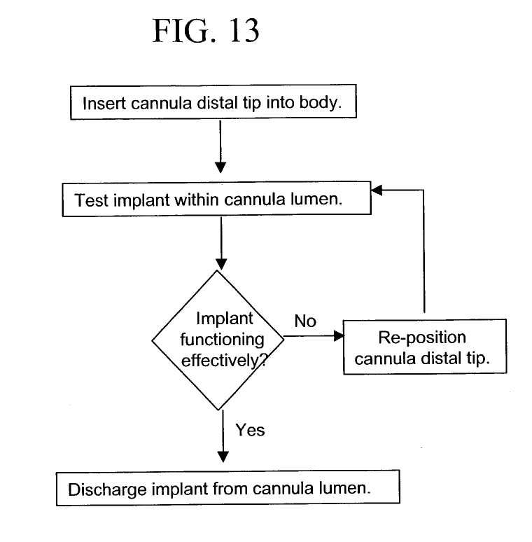

In one embodiment, the invention may include a method for positioning an implant in a body at a target location at which the implant will function effectively including: (a) inserting a cannula distal tip having the implant retained in the cannula lumen into the body until the implant reaches a testing position; (b) testing the implant while within the cannula lumen at the testing position to determine whether the implant is functioning effectively; (c) discharging the implant from the lumen of the cannula at the testing location if the testing reveals that the implant is functioning effectively at the test location. This method may be utilized to pre-test the implant itself at the testing position prior to releasing it from the injection device, as is depicted in FIG. 13.

In one embodiment, the method may further include moving the cannula containing the implant to a new test location, if testing shows that the implant is not located at an effective position, and re-testing the implant while within the cannula lumen at the new testing position to determine whether the implant is functioning effectively, as shown in dashed lines in FIG. 13. In some methods, movement of the implant to a new test location may comprise moving the implant longitudinally relative to the target location. In some methods, movement of the implant to a new test location may comprise rotating the implant axially relative to the target location.

In these embodiments testing of the implant may comprise any activity which is useful in assessing that the implant has been properly placed relative to the target tissue and/or that the implant is functioning effectively to achieve the desired result. In one embodiment, the implant is a microstimulator and testing of the implant may include delivery of a signal(s) to the microstimulator. In one example of this embodiment testing may consist of the delivery of a command signal to an implant from an external controller. Further, the command signal may be transmitted to the implant using electromagnetic radiation. Upon receipt of the command signal, the implant may generate an electrical stimulation current which is applied to the surrounding tissues via electrodes at the two ends of the implant. If the implant is correctly placed and functioning in or near a muscle or muscle nerve, the operator may observe the contraction thereby induced in the muscle, confirming the placement and function of the implant.

In one embodiment, the implant is a microstimulator and testing of the implant may include receipt and analysis of a signal from microstimulator. In one example of this embodiment testing may consist of the receipt and analysis of a reporting signal from an implant to an external controller. For example, an accelerometer that is sensitive to gravitational force will generate a signal proportional to the vector component of that force acting on the sensor depending on its three dimensional orientation in the body with respect to the gravitational vertical axis.

In one embodiment, the implant may sense the bioelectric signals produced by a muscle or nerve by means of electrodes affixed to the implant.

In another embodiment, the implant is a microstimulator and testing of the implant may include exposing the external electrode(s) of the microstimulator to interstitial fluids at the test location during testing. For example, where channels are formed within the cannula, interstitial fluid may contact external electrodes of the implant. This is advantageous at least in that the electrodes are in fluid communication with the target site and can therefore directly electrically stimulate or record from the environment of the target location while still contained in the injection device.

In one embodiment, the implant is discharged from the cannula lumen at the testing location by maintaining position of implant at testing location while cannula is withdrawn. Further, the longitudinal and/or axial position of the implant may be maintained relative to the testing location when the implant is discharged. For example, in discharging the implant a probe may be used to stabilize the implant while a cannula is withdrawn to expose the implant at the tested location.

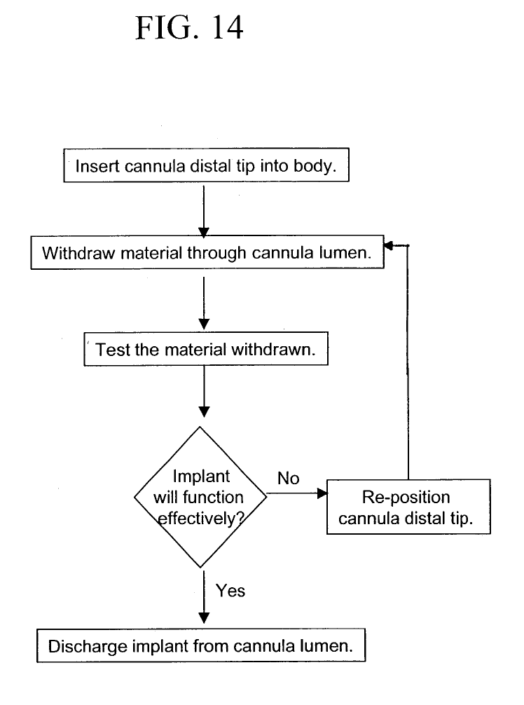

In one alternative embodiment, the invention may include a method for positioning an implant in a body at a target location at which the implant will function effectively including: (a) inserting a distal tip of a cannula having the implant retained within a lumen therein into the body until the tip reaches a testing position; (b) withdrawing material from the testing position through a communication channel extending from a distal end of the cannula proximate to the testing position to a proximal end of the cannula; (c)testing the material withdrawn from the testing position; (d) discharging the implant from the cannula lumen at the testing position if the testing shows that the implant will operate effectively at the test location. This method may be utilized to test the environment at the target site prior to releasing the implant from the injection device, and is depicted in FIG. 14.

In one embodiment, the method may further include moving the implant to a new location if testing shows that the implant is not located at an effective position or in a desirable environment, and re-testing the implant while within the cannula lumen at the testing position to determine whether the implant is in an effective position or desirable environment, as depicted in dashed lines in FIG. 14. In some embodiments, movement of the implant may comprise moving the implant longitudinally relative to the target location. In some embodiments, movement of the implant may comprise rotating the implant axially relative to the target location.

For example, attempts to withdraw material through the insertion tool may be useful to determine the presence or absence of an expected tissue/fluid at a desired target site. For example, testing may used to confirm that there is no hematoma at the target site. It may be undesirable to place an implant in a hematoma because the pool of fluid will interfere with its function and with its proper fixation in the target site and poses an increased risk of infection. The ability to withdraw material may be useful to determine the presence of free air if the lung or other hollow visceral organ has been punctured during insertion. Similarly the presence of another fluid such as cerebrospinal fluid, urine, etc. may signify an undesirable event or location of the insertion tool.

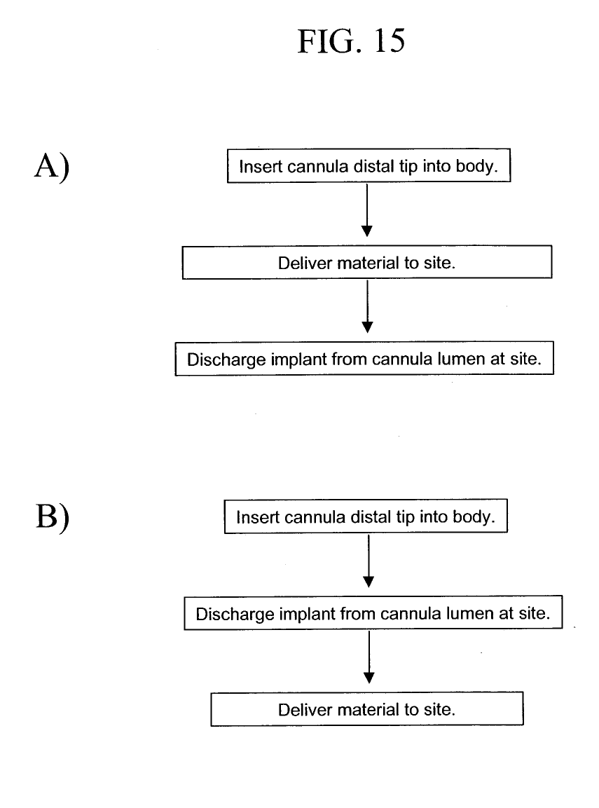

In one alternative embodiment, the invention may include a method for injecting material at the site of an implant in a body, including: (a) inserting a distal tip of a cannula having an implant retained within a lumen therein to a site within the body; (b) delivering material to the area of the site through a communication channel extending from a proximal end of the cannula to a distal end thereof; and (c) discharging the implant from the lumen of the cannula at the site. This method may be used to treat the target location prior to or after implant positioning, and is depicted in FIG. 15A.

Examples of materials which may be desirable to deliver to the target site include, but are not limited to steroids to limit peri-implant capsular formation around the implant.

Further, the embodiment may include testing the implant before delivering material to the site to determine whether the implant is functioning effectively. Further, if the implant is functioning effectively, then delivering the implant to the site. Further, if the implant is not functioning effectively, moving the implant to a new location and retesting or removing the implant if desired.

Further, the embodiment may include withdrawing material from the testing position, testing the material withdrawn from the testing position before delivering material to the site. Further,.if the testing shows that the implant will function effectively at the test location, then delivering the implant to the site. Further, if the testing shows that the implant will not function effectively at the test location, moving the implant to a new location and re-testing, or removing the implant if desired.

Alternatively, the invention may include a method for injecting material at the site of an implant in a body, including: (a) inserting a distal tip of a cannula having an implant retained within a lumen therein to a site within the body; (b) discharging the implant from the lumen of the cannula at the site; and (c) delivering material to the area of the site through a communication channel extending from a proximal end of the cannula to a distal end thereof, as depicted in FIG. 15B. As described above, this method can also be combined with other methods of using the injection device. For example, drugs or hormones such as anabolic steroids could be injected into the site where an electrical stimulator is implanted in order to modulate or augment the trophic response of muscles to the electrical activation. Other examples include other steroids, anti-inflammatory agents, antibiotics, and analgesics.

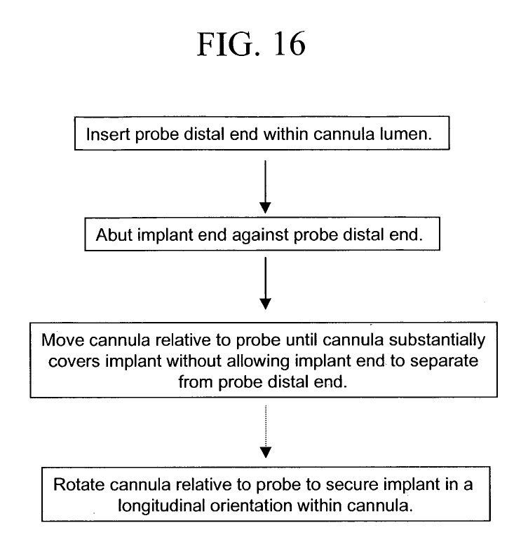

In one alternative embodiment, the invention may include a method of loading an implant having an implant end into an injection device including a cannula, and a probe having a distal end sized to fit within the cannula lumen having a distal end, the method including: (a) inserting the probe distal end within the cannula lumen; (b) abutting the implant end against the distal end of the probe; and (c) moving the cannula relative to the probe until the cannula substantially covers the implant without allowing the implant end of the implant to separate from the probe distal end, as depicted in FIG. 16.

In one embodiment, the method may further comprise rotating cannula relative to a probe to secure the implant in a longitudinal orientation within the cannula, as depicted in dashed lines at FIG. 16B.

The channels in the cannula wall, the cannula distal end, arrangement of the probe and cannula handles, probe lumen, travel groove on probe and syringe port all contribute to providing thorough access for sterilization of the injection device by autoclaving or other suitable methods.

Implant positioning using the devices, implants and methods of this invention may be used in combination with existing methods practiced in the art, such as fluoroscopy, CT and ultrasound to visualize the implant relative to target structures in the body.

The injection device and methods described could be modified for use with any implant of any size or shape suitable for injection into a target location in the body. Further, any item may be configured for delivery using the injection device and methods described herein by being placed in a capsule configured for use in this invention.

While the specification describes particular embodiments of the present invention, those of ordinary skill can devise variations of the present invention without departing from the inventive concept. For example, any of the structural embodiments may be combined to form an injection device of this invention. Further, any of the methods may be combined to use the invention.

We claim:

1. A method for positioning an implant in a body at a target location at which the implant will function effectively comprising:
a) inserting a distal tip of a cannula having the implant retained within a cannula lumen into the body until the implant reaches a testing position;
b) testing the implant while within the cannula lumen at the testing position to determine whether the implant is functioning effectively;
c) discharging the implant from the lumen of the cannula at the testing location if the testing reveals that the implant is functioning effectively at the test location.

2. The method of claim 1, further including moving the implant within the cannula lumen to a new position if testing shows the implant is not located at effective position and re-testing the implant while within the cannula lumen at the testing position to determine whether the implant is functioning effectively.

3. The method of claim 2 wherein the movement of the implant includes longitudinal movement relative to the target location.

4. The method of claim 2 wherein the movement of the implant includes axial rotation relative to the target location.

5. The method of claim 1, wherein the implant is a microstimulator.

6. The implant of claim 1, wherein the implant includes a dissolvable capsule.

7. The method of claim 1 wherein the implant is a microstimulator, and wherein testing includes the delivery of a signal to the microstimulator.

8. The method of claim 1, wherein the implant is a microstimulator, and wherein testing includes the receipt and analysis of a signal from the microstimulator.

9. The method of claim 1, wherein the implant is a microstimulator, and wherein testing includes determining whether the microstimulator is stimulating the target location.

10. The method of claim 1, wherein the implant is a microstimulator having at least one external electrode, and wherein the external electrode is exposed to interstitial fluid of the target location during testing.

11. The method of claim 1, wherein the implant is maintained at the testing location during the discharge of the implant from the lumen.

12. The method of claim 11, wherein the implant is maintained in that same axial orientation relative to the testing location.

13. The method of claim 11, wherein the implant is maintained in that same longitudinal orientation relative to the testing location.

14. A method for positioning an implant in a body at a target location at which the implant will function effectively, comprising:
a) inserting a distal tip of a cannula having the implant retained within a cannula lumen into the body until the tip reaches a testing position;
b) withdrawing material from the testing position through a lumen extending from a cannula distal end proximate to the testing position to a cannula proximal end;
c) testing the material withdrawn from the testing position;
d) discharging the implant from the cannula lumen at the testing position if the testing shows that the implant will operate effectively at the test location.

15. The method of claim 14, further including moving the implant within the cannula lumen to a new position if testing shows the implant is not located at effective position and withdrawing material from the testing position through a lumen extending from a cannula distal end proximate to the testing position to a cannula proximal end; and testing the material withdrawn from the new testing position.

16. The method of claim 14, wherein the implant is a microstimulator.

17. The implant of claim 14, wherein the implant includes a dissolvable capsule.

18. The method of claim 14, wherein the testing of the material from the testing position is to determine the presence or absence of blood.

19. A method for injecting material at the site of an implant in a body, comprising:
a) inserting a distal tip of a cannula having an implant retained within a cannula lumen to a site within the body;
b) delivering material to the area of the site through a lumen extending from a cannula proximal end to a cannula distal end; and
c) discharging the implant from the cannula lumen at the site.

20. The method of claim 19, wherein the material is a selected from the group comprising steroids, anti-inflammatory agents, antibiotics, analgesics.

21. The method of claim 19, testing the implant while within the cannula lumen at the testing position to determine whether the implant is functioning effectively prior to delivering material to the area.

22. The method of claim 19, wherein the implant is a microstimulator.

23. The implant of claim 19 wherein the implant includes a dissolvable capsule.

24. A method for injecting material at the site of an implant in a body, comprising:
a) inserting a distal tip of a cannula having an implant retained within a cannula lumen to a site within the body;
b) discharging the implant from the cannula lumen at the site;
c) delivering material to the area of the site through a lumen extending from a cannula proximal end to a cannula distal end.

25. The method of claim 24, testing the implant while within the cannula lumen at the testing position to determine whether the implant is functioning effectively prior to discharging the implant from the cannula site.

26. The method of claim 24, wherein the material is a selected from the group comprising steroids, anti-inflammatory agents, antibiotics, or analgesics.

27. The method of claim 24, wherein the implant is a microstimulator.

28. The implant of claim 24, wherein the implant includes a dissolvable capsule.

29. A method of loading an implant having an end into an injection device used to inject the loaded implant into a body, the injection device including a cannula with a cannula lumen and a probe sized to fit within the lumen having a distal end, the method comprising:
a) inserting the probe distal end within the cannula lumen;
b) abutting the implant end of the implant against the probe distal end;
and
c) moving the cannula relative to the probe until the cannula substantially covers the implant without allowing the implant end to separate from the probe distal end.

30. The method of claim 29, further including rotating the probe relative to cannula.

31. The method of claim 29, wherein the implant is a microstimulator.

32. The implant of claim 29, wherein the implant includes a dissolvable capsule.

33. An injection device for positioning an implant in a body at a target location at which the implant will function effectively comprising:
a) a cannula having a cannula lumen;
b) an implant positioned within the cannula lumen;
c) a surface at the end of the implant configured to releasably engage a surface within the cannula lumen.

34. The injection device of claim 33, wherein the cannula lumen includes a cannula detent and the implant includes an annular ring having a notch therein, wherein the notch is configured to move slidable along the detent, when the detent and notch are axially aligned in the cannula lumen.

35. The injection device of claim 33, wherein the cannula lumen includes a cannula detent and the implant includes an retaining member comprising a post and an annular ring, wherein the length of the post is configured to permit the detent to rest within a detent space formed between the implant end surface and the annular ring, and wherein the annular ring includes a notch is configured to move slidable along the detent, when the detent and notch are axially aligned in the cannula lumen.

36. The injection device of claim 33, wherein the injection device further includes a probe positioned substantially within the lumen and having a probe distal end; and a surface at the end of the implant abutting a surface at the probe distal end of, wherein both the implant end surface and probe distal end surface are configured to prevent the implant from rotating with respect to the probe while the surfaces abut.

37. The injection device of claim 33, wherein the injection device wherein the implant includes at least one implant external electrode positioned within the cannula lumen; and the injection device includes at least one fluid communication channel within the cannula wall substantially aligned with the implant external electrode.

38. The injection device of claim 33, wherein the cannula includes a distal end formed into a trochar.

39. The injection device of claim 33, wherein the cannula is comprised of a material that will not shield/interfere with electromagnetic signals.

40. The injection device of claim 33, wherein the cannula is comprised of electrically insulating material.

41. The injection device of claim 33, wherein the implant is a microstimulator.

42. The injection device of claim 33, wherein the implant includes a dissolvable capsule.

43. An injection device for positioning an implant in a body at a target location at which the implant will function effectively comprising:
a) a cannula having a cannula lumen;
b) a probe positioned substantially within the cannula lumen and having a probe distal end;
c) an implant positioned within the cannula lumen;
d) a surface at the end of the implant abutting a surface at the distal end of the probe, the implant end surface and the probe end surface being configured to prevent the implant from rotating with respect to the probe while the implant end surface and probe end surface abut.

44. The injection device of claim 43, wherein the implant end surface and probe end surface are further configured to permit the implant to separate longitudinally from the probe.

45. The injection device of claim 43, wherein the implant end surface includes a slot and the probe end surface includes tab.

46. The injection device of claim 43, wherein the implant end surface includes a tab and probe end surface includes a slot.

47. The injection device of claim 43, wherein the cannula further includes a cannula handle at a cannula proximal end, and wherein the probe further includes a probe handle at a probe proximal end, wherein the probe handle is configured to move longitudinally within a lumen in the cannula handle and wherein the cannula handle and probe handle are configured to permit defined longitudinal movement of the cannula relative to the probe.

48. The injection device of claim 43, wherein the cannula further includes a cannula handle at a cannula proximal end, and wherein the probe further includes a probe handle at a probe proximal end, wherein the probe handle is configured to move longitudinally within a lumen in the cannula handle and wherein the cannula handle and probe handle are configured to permit defined axial movement of the cannula relative to the probe.

49. The injection device of claim 43, wherein the implant end surface is further configured to releasably engage a surface within the lumen.

50. The injection device of claim 43, wherein the implant includes at least one implant external electrode positioned within the cannula lumen; and the injection device includes at least one fluid communication channel within the cannula wall substantially aligned with the implant external electrode.

51. The injection device of claim 43, wherein the cannula includes a distal end formed into a trochar.

52. The injection device of claim 43, wherein the cannula is comprised of a material that will not shield/interfere with electromagnetic signals.

53. The injection device of claim 43, wherein the cannula is comprised of electrically insulating material.

54. The injection device of claim 43, wherein the implant is a microstimulator.

55. The injection device of claim 43, wherein the implant includes a dissolvable capsule.

56. An injection device for injecting an implant in a body at a target location at which the implant will function effectively comprising:
a) a cannula having a cannula wall forming a cannula lumen, and a cannula distal end formed into a trochar;
b) an implant releasably engaged within the cannula.

57. The injection device of claim 56, further including an apparatus for assisting in the release of an implant into the body at the target location.

58. The injection device of claim 56, wherein the cannula has a slit formed the cannula wall to form a cannula upper casing and a cannula lower casing, wherein the slit extends from the cannula distal end for a distance greater than a length of the implant.

59. The injection device of claim 56, wherein the implant includes an implant end surface is configured to releasably engage a surface within the cannula lumen.

60. The injection device of claim 56, wherein the injection device further includes:
a) a probe positioned substantially within the cannula lumen and having a probe distal end;
b) a surface at the end of the implant abutting a surface at the probe distal end, wherein the implant end surface and the probe end surface are configured to prevent the implant from rotating with respect to the probe while the implant end surface and probe end surface abut.

61. The injection device of claim 56, wherein the implant includes at least one implant external electrode positioned within the cannula lumen; and the injection device includes at least one fluid communication channel within the cannula wall substantially aligned with the implant external electrode.

62. The injection device of claim 56, wherein the cannula is comprised of a material that will not shield/interfere with electromagnetic signals.

63. The injection device of claim 56, wherein the cannula is comprised of electrically insulating material.

64. The injection device of claim 56, wherein the implant includes a dissolvable capsule.

65. The injection device of claim 56, wherein the implant is a microstimulator.

66. An implant configured to be injected by an injection device into body tissue or a body cavity and configured with at least one surface that releasably interlocks with a surface in the injection device.

67. The implant of claim 66, wherein the implant interlocking surface is configured to maintain longitudinal alignment between the implant and the injection device while the capsule is contained within the injection device.

68. The implant of claim 66, wherein the implant interlocking surface is configured to maintain axial alignment between the implant and the injection device while the capsule is contained within the injection device.

69. The implant of claim 68 wherein the implant interlocking surface is configured to allow the implant to be separated longitudinally from the injection device after during implantation.

70. The injection device of claim 66, wherein the implant is a microstimulator.

71. The implant of claim 66, wherein the implant includes a dissolvable capsule.

72. The implant of claim 66 wherein the capsule comprises polyglactic acid or polydioxanone or a combination of polyglactic acid or polydioxanone.

73. An injection device comprising:
a) a cannula having a cannula wall forming a cannula lumen, wherein the cannula lumen includes a detent, and wherein the cannula wall includes at least one channel therein;
b) a probe configured to move slidably within the cannula lumen, and wherein the probe includes a tab at the probe distal end;
c) an implant including a retaining member, wherein the retaining member includes a notch configured to move slidably along the detent when the implant is rotated axially with respect to the cannula lumen; and
d) the implant further including a surface configured to be releasable connected to the probe tab such that rotational, but not longitudinal movement between the probe and implant is prevented while the implant surface and tab are connected.

74. The injection device of claim 73, wherein the implant is a microstimulator.

75. The injection device of claim 73, wherein the implant includes a dissolvable capsule.
